(19) United States
(12) Patent Application Publication
(10) Pub. No.: US 2005/0137122 A1
(43) Pub. Date: Jun. 23, 2005

(54) Use of agents that prevent generation of amyloid and amyloid-like lipoproteins, and/or use of agents that promote sequestration and/or degradation of, and/or prevent neurotoxicity of such proteins in the treatment of hearing loss and improving body balance

(75) Inventor: Najam A. Sharif, Keller, TX (US)

Correspondence Address: Teresa J. Schultz;Alcon Research, Ltd., 6201 S. Freeway, Mail Code Q-148, Fort Worth, TX, 76134-2099, US

(73) Assignee: Alcon, Inc.

(21) Appl. No.: 11/005,923

(22) Filed: Dec. 7, 2004

Related U.S. Application Data
(60) Provisional application No. 60/530,434, filed on Dec. 17, 2003.

(51) Int. Cl. A61K 38/17; A61K 31/7076; A61K 31/553; A61K 31/353; A61K 31/20

(52) U.S. Cl. 514/2; 514/440; 514/49; 514/558; 514/571; 514/450

(57) ABSTRACT

The present invention provides compositions and methods for treating otic disorders. More specifically, the present invention describes the use of agents that down-regulate expression of Tanis and/or p21^{Waf1/Cip1/Sd1} genes to treat such disorders of the ear.

### BACKGROUND OF THE INVENTION

1. Field of the Invention

The present invention relates to the field of treatment of hearing loss and body imbalance. More particularly, the present invention relates to the treatment of hearing loss and body imbalance by administering to a patient suffering therefrom an amount of a compound that prevents the generation and deposition of amyloid and amyloid-like proteins, and/or promotes the sequestration and/or degradation of amyloid proteins and/or prevents the neurotoxic effects of such proteins as and when they are generated and deposited in the middle and/or inner ear and/or on or along the otic and vestibular nerves projecting to the brain. The present invention also provides compositions and methods for treating the afore-mentioned otic disorders by sequestering and/or degrading Tanis gene product protein (TGPP) and/or p21^{Waf1/Cop1/Sdi1} gene product protein (p21GPP) in otic tissues. In addition, compositions and methods to prevent the generation of TGPP and/or p21GPP and/or to prevent the neurotoxic effects of such gene product proteins are provided to treat the otic disorders. In addition, agents that stop or reduce the initial activation of Tanis and p21^{Waf1/Cop1/Sdi1} genes and/or prevent nerve cell death due to the presence of TGPP or p21GPP would also be useful to treat the patient's otic disorders.

2. Description of the Related Art

There are a number of otic (connected with the ear) conditions that are caused by, or aggravated by, damage to the otic nerve and/or degeneration of otic tissues and the nerve, and/or degeneration of the inner ear hair cells that transmit messages via the otic nerve to the brain hearing center, and/or elevated otic pressure. Otic disorders include hearing loss, problems with maintaining good body balance and ringing in the ear (tinnitus) that can result from different types of insults (see below).

The ear is highly innervated with sensory afferents and efferents capable of receiving and transmitting various messages connected with the hearing sensation and s body balance status to the brain. The ear is comprised of outer, middle and inner ear portions and otic inflammation, edema, otic congestion, otic pressure, infection, accidental trauma, surgical procedures and post-surgical recovery can cause rapid hearing loss and/or sensation of balance problems.

The outer or “external” ear is comprised of the pinna and external ear canal (“EAC”). The EAC is a tubular, slightly curved structure extending from the pinna to the tympanic membrane or “ear drum.” Sound travels through the EAC and causes the tympanic membrane to vibrate. Various disorders can arise in the outer ear eliciting pain to the host. For example, otitis externa is an acute, painful inflammatory condition of the EAC that affects all age groups of humans and accounts for roughly half of the ear pain pathologies known to exist. During the summer months, cases of otitis externa tend to increase due to what is known as “swimmer's ear.” Swimmer's ear generally arises from the seepage of water into the EAC during swimming and the onset of infection and pain. Other outer ear disorders causing pain to the host include insertion of foreign objects in the ear, cerumen impaction, long-term use of hearing aids, and dermatological disorders, including psoriasis, eczema and seborrhea.

The middle ear is an air-filled cavity between the outer and inner ears. The middle ear is separated from the outer ear by the tympanic membrane and abuts the inner ear. It has a volume of about two milliliters and is connected to the back of the throat via the eustachian tube. The middle ear contains the malleus, icus and stapes, which are tiny bones that translate the movement of the tympanic membrane (arising from sound waves received from the outer ear) to the inner ear. Various conditions of the middle ear can cause pain to the host. For example, otitis media (OM), which can be acute (“AOM”) or associated with effusion (“OME”), is an inflammatory condition of the middle ear which generally affects children more often than adults (Karver, Otitis Media, Primary Care, Volume 25, No. 3, pages 619-632 (1998). The etiology of otitis media is fairly broad and can be caused by various inflammatory events including infection and allergy. Effusion, which can be sterile or contain infectious material, may also result from otitis media. This fluid consists of various inflammatory cells (white blood cells), mediators of allergy and inflammation and cellular debris.

The inner ear comprises the sensory organs of the auditory and vestibular systems. It consists of two major compartments, known as the bony and membranous labyrinths. These chambers are highly organized and sensitive tissues and provide both auditory perception and balance to the animal. Various pathologies may arise in the inner ear, creating distortion of hearing, loss of balance and pain.

For instance, since otic pain is often associated with infection and resultant congestion and pressure, the primary therapeutic approach to treating otic pain is the administration of antiobiotics, both systemically and topically. Various other therapies have been attempted for the alleviation of otic pain. Topical steroids (e.g., hydrocortisone) and systemic non-steroidal anti-inflammatory drugs (NSAIDs), such as aspirin and ibuprofen, have been used typically in conjunction with anti-infectives to treat otic pain. Local anesthetics are another class of compounds which relieve pain by directly inhibiting nerve cellular function. A drawback of local anesthetic therapy is the short duration of action of such drugs. Another problem with the use of local anesthetics is that their mechanism of action, non-specific membrane stabilization, can have the undesired coincident effect of also inhibiting biological functions of cells, such as fibroblasts and surrounding neural cells. Topical steroids have their own attendant side-effects as well. Therefore, even though pain sensation can be abated with local anesthetic treatment, healing and normal function of the tissue may be significantly compromised.

If the allergic inflammatory and infective conditions of the ear are not treated in a timely manner different degrees of hearing loss and body imbalance can ensue. These problems may results from congestion and elevated otic pressure due to the edema and accumulation of inflammatory cells (white blood cells), mediators of allergy and inflammation and cellular debris in the different parts of the ear.

Long term hearing loss may also result directly from the afore-mentioned conditions coupled with or due to the generation and deposition of lipoproteins such as amyloid proteins in the middle and inner ear compartments and on/or around the hair cell terminals and/or around the otic nerve head. Such abnormal accumulation of amyloid and/or amyloid-like lipoproteins have been reported to be associated with the etiology of many age-related and inflammatory conditions such as atherosclerotic cardiovascular disease (Urieli et al., 1998), arthritis (O'Hara et al., 2000), Alzheimer's disease (Liang et al., 1997; Caricasole et al. 2003; Kindy et al., 1999), age-related macular degeneration associated with the retina (Ambati et al., 2003), and glaucoma associated with elevated ocular pressure in the anterior eye segment due to extracellular matrix accumulation and/or optic nerve head damage due to the latter and other degenerative processes at the back of the eye (Schwartz et al., 1982; Ermilov et al., 1993; Krasnov et al., 1996). Accordingly, if the neurotransmission between inner ear hair cells and the otic nerve-head is compromised by the accumulation of such lipoproteins or due to the other factors mentioned above, or the otic nerve itself is damaged, begins to degenerate or is compromised in other ways, then suitable therapeutic intervention is necessary to prevent or at least reduce the potential for hearing loss and balance/equilibrium problems. Another problem that can result from trauma and/or accumulation of extracellular debris like amyloids in the different parts of the ear and/or from over stimulation of the otic and vestibular nerves is the “ringing in the ear syndrome” called tinnitus. Tinnitus or t. aurium is the sensation of sound (ringing, whistling, booming) in one or both ears usually associated with disease in the middle ear, the inner ear, or the central auditory apparatus. Therefore, there is a medical need for suitable therapies to prevent/reduce the generation and deposition of amyloid proteins, to promote the degradation and/or sequestration of such amyloid proteins, to prevent or reduce the potential meachanical distortion of the nerve endings of the hair cells/otic and vestibular nerves by these amyloid proteins and further to prevent or reduce the potential neurotoxic effects of such lipoproteins at these various sites as has been reported for amyloids and their fragments in the brain (Lambert et al. 1998; Liu and Schubert, 1997; Nakagami and Oda, 2002a,b; Pike et al. 1993; Zhang et al., 2001) and in the retina (Jen et al. 1998).

### SUMMARY OF THE INVENTION

Compositions and methods to prevent the generation of amyloid and/or amyloid-like proteins and/or to prevent the neurotoxic effects of such proteins are provided to treat tinnitus, hearing loss and to correct body imbalance and equilibrium associated with deposition of such amyloid proteins in various ear compartment and/or on or near the nerve endings of the otic and vestibular nerves. In one aspect, the present invention provides a method for treating these ear disorders by administering to a patient in need thereof a therapeutically effective amount of a composition comprising an agent that sequesters amyloid proteins in otic tissues and/or an agent that degrades amyloid proteins in otic tissues. The sequestration and/or degradation modulates the expression of the amyloid proteins, such that the patient's condition is treated. In addition, agents that stop or reduce the initial production of the amyloid proteins, and/or prevent the nerve cell death due to the presence of amyloid proteins would also be useful to treat the patient's otic disorders. In preferred embodiments, the agent will be a small molecular weight organic is molecule, antibody, protein, peptide, peptidomimetic, or nucleic acid.

The present invention also provides compositions and methods for treating otic disorders by sequestering and/or degrading Tanis gene product protein (TGPP) and/or p₂₁^{Waf1/Cop1/Sdi1} gene product protein (p21GPP) in otic tissues. In addition, compositions and methods to prevent the generation of TGPP and/or p21GPP and/or to prevent the neurotoxic effects of such gene product proteins are provided to treat the otic disorders mentioned above. In addition, agents that stop or reduce the initial activation of Tanis and p₂₁^{Waf1/Cop1/Sdi1} genes and/or prevent nerve cell death due to the presence of TGPP or p21GPP would also be useful to treat the patient's otic disorders.

Compounds that may be useful for preventing the production of amyloid and amyloid-like proteins include: γ-secretase inhibitors such as talsaclidine (Amyloid: J Prot. Fold. Disorder: 10, 1-6 [2003]), Xanomeline, L-689660, L-685458, McN-A-343, CDD-0097, fenchylamine, MG132, WPE-111-31C, MW-11-36C/26A, MW-167, CM-265, lactacystin, DNPS1 and DAPT (J. Pharamacol. & Expt. Ther. 305: 864, 2003). Other compounds of use may include the statin family, e.g. pravastatin, atorvastatin (see Neurochem Res. 28: p.979-986 & p.1049-1062 [2003]) and presenilinase inhibitors such as pepstatin A (Drug News Perspect. 16: 69 [2003]) and talsaclidine (Amyloid: J Prot. Fold. Disorder: 10, 1-6 [2003]).

Compounds that may be useful for promoting degradation of amyloids and related proteins include glycoaminoglycans and congo red (J. Neurochem. 70: 292-298 [1998]).

Compounds that may be useful for promoting sequestration or clearance of amyloids and related proteins include gelsolin and ganglioside GM1 (J. Neurosci. 23: 29-33 [2003]). In addition, antibodies raised against amyloid proteins and/or against amyloid-like proteins would be useful for sequestration and clearance of the former detrimental proteins as has been shown in the brain (Nature, 400: 173-177 [1999]; Nature , 408: 979-982 [2000]; Nature, 408: 982-985 [2000]).

Compounds that may be useful for preventing or diminishing the neurotoxic effects of amyloids and related proteins include: RS-0466 (Eur. J. Pharmacol. 457: 11-17 [2002]; Br. J. Pharmacol. 137: 676-682 [2002]), V-type ATPase inhibitors (bafilomycin and concanamycin; J. Neurochem. 72: 1939-1947 [1999]), tachykinin peptides and their non-peptide analogs (Science 250: 279-282 [1990]), α-lipoic acid (Neurosci. Lett. 312: 125-128, 2001), propentofylline (Eur. J. Pharmacol. 458: 235-241 [2003]), glycogen synthase kinase-3β (GSK-3β) inhibitors (Trends Mol. Med. 8: 126-132, 2002; Trends Pharmacol. Sci. 24: 233-238 [2003]), memantine (Neuropharmacol. 38: 1253-1259 [1999]), mixed cyclin-dependent kinase-GSK3β inhibitors (Oncogene, 20: 3786-3797 [2001]), COX-2 inhibitors (Neurobiol. Aging, 23: 327-334 [2002]) and propentofylline (Eur. J. Pharmacol. 458: 235-241 [2003]).

The present invention further provides compositions for treating otic disorders by administering a composition containing a p21^{Waf1/Cip/Sdi1} gene product protein (see below) inhibitor and/or inhibitors of cyclin dependent kinase-1 (CDK1), CDK2, CDK5 and CDK9, and inhibitors of cJAK and ASRK-1 including the following agents: olomoucine, roscovitine, purvalanol, kenpaullone, alsterpaullone, indirubins, flavopiridol, stauroporine and analogs and derivatives of the above compounds.

### DETAILED DESCRIPTION PREFERRED EMBODIMENTS

Human serum amyloid A (SAA) comprises a number of small, differentially expressed apolipoproteins encoded by genes localized on the short arm of chromosome 11. There are four isoforms of SAAs. SAA1 (SEQ ID NO:2), encoded by SEQ ID NO: 1, and SAA2 (SEQ ID NO:4), encoded by SEQ ID NO:3, are known as acute phase reactants, like C-reactive protein, that is, they are dramatically upregulated by proinflammatory cytokines. The 5′UTR promoter regions of SAA1 and SAA2 genes are also provided (SEQ ID NO:12 and SEQ ID NO:13, respectively). SAA3 (SEQ ID NO:5) is a pseudogene and SAA4 (SEQ ID NO:6) is a low level endogenously expressed gene encoding endogenous SAA (SEQ ID NO:7). SAA2 has two isoforms, SAA2α (SEQ ID NO:9) and SAA2β (SEQ ID NO: 11), which differ by only one amino acid. SAA1 and SAA2 proteins are 93.5% identical at the amino acid level (SEQ ID NO:2 and SEQ ID NO:4, respectively) and these genes are 96.7% identical at the nucleotide level (SEQ ID NO: 1 and SEQ ID NO:3, respectively).

SAA is an acute-phase reactant whose level in the blood is elevated approximately 1000-fold as part of the body's responses to various injuries, including trauma, infection, inflammation, and neoplasia. As an acute-phase reactant, the liver has been considered to be the primary site of expression. However, extrahepatic SAA expression was described initially in mouse tissues, and later in cells of human atherosclerotic lesions (O'Hara et al. 2000). Subsequently, SAA mRNA was found widely expressed in many histologically normal human tissues. Localized expression was noted in a variety of tissues, including breast, stomach, small and large intestine, prostate, lung, pancreas, kidney, tonsil, thyroid, pituitary, placenta, skin epidermis, and brain neurons. Expression was also observed in lymphocytes, plasma cells, and endothelial cells. SAA protein expression co-localized with SAA mRNA expression has also been reported in histologically normal human extrahepatic tissues. (Liang et al. 1997; Urieli-Shoval et al. 1998). SAA gene expression is elevated significantly in glaucomatous TM tissues. Increased SAA may be involved in the generation of elevated intraocular pressure and damage to the optic nerve leading to vision loss in glaucoma patients (U.S. application Ser. No. 60/530,430). Even though the ear compartments and otic and vestibular nerve-heads and axons were not studied by the different groups cited above, it is anticipated that the phenomenon of abnormal generation and deposition of SAA and other amyloid proteins in the otic system and associated brain areas would also occur as a result of normal aging process and be accelerated during traumatic, inflammatory and infective conditions of the ear.

SAA isoforms are apolipoproteins that become a major component of high-density lipoprotein (HDL) in the blood plasma of mammals and displaces A-I (ApoA-I) and phospholipid from the HDL particles (Miida et al. 1999). SAA binds cholesterol and may serve as a transient cholesterol-binding protein. In addition, over-expression of SAA1 or SAA2 leads to the formation of linear fibrils in amyloid deposits, which can lead to pathogenesis (Uhlar and Whitehead 1999; Liang et al. 1997). SAA plays an important role in infections, inflammation, and in the stimulation of tissue repair. SAA concentration may increase up to 1000-fold following inflammation, infection, necrosis, and decline rapidly following recovery. Thus, serum SAA concentration is considered to be a useful marker with which to monitor inflammatory disease activity. Hepatic biosynthesis of SAA is up-regulated by pro-inflammatory cytokines, leading to an acute phase response.

Chronically elevated SAA concentrations are a prerequisite for the pathogenesis of secondary amyloidosis, a progressive and sometimes fatal disease characterized by the deposition in major organs of insoluble plaques composed principally of proteolytically cleaved SAA. This same process also may lead to atherosclerosis. There is a requirement for both positive and negative SAA control mechanisms to maintain homeostasis. These mechanisms permit the rapid induction of SAA expression to fulfill host-protective functions, but they also must ensure that SAA expression is rapidly returned to baseline levels to prevent amyloidosis. These mechanisms include modulation of promoter activity involving, for example, the inducer nuclear factor kB (NF-kB) and its inhibitor IkB, up-regulation of transcription factors of the nuclear factor for interleukin-6 (NF-IL6) family, and transcriptional repressors such as yin and yang 1 (YY1). Post-transcriptional modulation involving changes in mRNA stability and translation efficiency permit further up- and down-regulatory control of SAA protein synthesis to be achieved. In the later stages of the AP response, SAA expression is effectively down-regulated via the increased production of cytokine antagonists such as the interleukin-1 receptor antagonist (IL-1Ra) and of soluble cytokine receptors, resulting in less signal transduction driven by pro-inflammatory cytokines (Jensen and Whitehead, 1998).

The Tanis gene (SEQ ID NO: 14) is a recently identified gene that encodes a membrane protein (SEQ ID NO:15) said to bind to SAA (Walder et al. 2002). It is believed that therapeutic intervention of the interaction between SAA and its putative receptor, encoded by the Tanis gene, may modulate SAA expression levels and/or receptor-mediated SAA signaling. Methods for the identification of agents that interfere with this interaction and their use for the treatment of otic disorders are also provided herein.

It has also recently been discovered that a gene called p21^{Waf1/Cip/Sdi1} (SEQ ID NO:16) activates SAA and activates the gene APP that produces amyloid protein which forms plaques in the brain that are hallmarks of Alzheimer's disease (Chang et al, 2000; Kindy et al., 1999; Johan et al, 1997). In addition, p21^{Waf1/Cip/Sdi1}-induced gene expression results in over production of extracellular matrix (ECM) proteins including fibronectin-1, plaminogen activator inhibitor, tissue-type plasminogen activator, integrin β3 (Chang et al., 2000) which may be contributive factors in the glaucomatous situation. Likewise, p21^{Waf1/Cip/Sdi1}-induced connective tissue growth factor and galectin-3 (Chang et al., 2000) may also play significant roles in deposition of ECM proteins and other components of ECM in the otic compartments leading to the ear disorders emntioned above. Therefore, it is believed that inhibition of p21^{Waf1/Cip/Sdi1} gene would be useful in the treatment of the pathophysiology of otic disorders. Interestingly, since p21^{Waf1/Cip/Sdi1} gene expression results in natural inhibition of cyclin-dependent kinases (CDK) (Chang et al. 2000), and since p21^{Waf1/Cip/Sdi1} was reported to bind c-Jun amino-terminal kinase (cJAK), apoptosis-signal-regulating kinase 1 (ASRK-1) and Gadd45 (Chang et al. 2000), it follows that inhibitors of these kinases would also act as the p21^{Waf1/Cip/Sdi1} gene. Accordingly, inhibitors of CDK1,2,5 and 9, and inhibitors of cJAK and ASRK-1 would be useful for treating ocular hypertension, glaucoma and ARMD. Agents which may modulate the interaction of SAA and its putative receptor and the TGPP or p21GPP include, but are not limited to, peroxisome proliferator-activated receptor α (PPARα) agonists, tachykinin peptides and their non-peptide analogs, and α-lipoic acid. PPARα agonists include arachidonic acid, linoleic acid, docosahexaenoic acid, eicosapentaenoic acid, 8(S)-HETE, (±)ibuprofin, indomethacin, leukotriene B₄, meclofenamate, prostaglandin A₁, prostaglandin A₂, prostaglandin D₁, prostaglandin D₂, prostaglandin J₂, 15-deoxy-Δ¹²-prostaglandin J₂, WY 14643, ciglitizone, carbaprostacyclin and prostacyclin. Examples of preferred agents for use in the present invention include fenofibrate, WY 14643, (4-chloro-6-(2,3-xylidino)-2-pryrimidinylthiol)-acetic acid), ciprofibrate, 2-bromohexadecanoic acid, bezafibrate, ciglitizone, bafilomycin, concanamycin, deprenyl and desmethyldeprenyl.

The present invention provides methods and compositions of using agents that sequester and/or degrade amyloids or amyloid-like proteins, agents that prevent or reduce the production of such proteins and/or agents that prevent or reduce the toxic effects of such proteins for the treatment of loss of body balance, reduction or loss of hearing and tinnitus in order to maintain and preserve the auditory and balance functions of the ear.

Compounds that may be useful for preventing the production of amyloid and amyloid-like proteins include: γ-secretase inhibitors such as talsaclidine (Amyloid: J Prot. Fold. Disorder: 10, 1-6 [2003]), Xanomeline, L-689660, L-685458, McN-A-343, CDD-0097, fenchylamine, MG132, WPE-111-31C, MW-11-36C/26A, MW-167, CM-265, lactacystin, DNPS1 and DAPT (J. Pharamacol. & Expt. Ther.305: 864, 2003). Other compounds of use may include the statin family, e.g. pravastatin, atorvastatin (see Neurochem Res. 28: p.979-986 & p.1049-1062 [2003]) and presenilinase inhibitors such as pepstatin A (Drug News Perspect. 16: 69 [2003]) and talsaclidine (Amyloid: J Prot. Fold. Disorder: 10, 1-6 [2003]).

Compounds that may be useful for promoting degradation of amyloids and related proteins include glycoaminoglycans and congo red (J. Neurochem. 70: 292-298 [1998]).

Compounds that may be useful for promoting sequestration or clearance of amyloids and related proteins include gelsolin and ganglioside GMI (J. Neurosci. 23: 29-33 [2003]). In addition, antibodies raised against amyloid proteins and/or against amyloid-like proteins would be useful for sequestration and clearance of the former detrimental proteins as has been shown in the brain (Nature, 400: 173-177 [1999]; Nature , 408: 979-982 [2000]; Nature, 408: 982-985 [2000]).

Compounds that may be useful for preventing or diminishing the neurotoxic effects of amyloids and related proteins include: RS-0466 (Eur. J. Pharmacol. 457: 11-17 [2002]; Br. J. Pharmacol. 137: 676-682 [2002]), V-type ATPase inhibitors (bafilomycin and concanamycin; J Neurochem. 72: 1939-1947 [1999]), tachykinin peptides and their non-peptide analogs (Science 250: 279-282 [1990]), α-lipoic acid (Neurosci. Lett. 312: 125-128, 2001), propentofylline (Eur. J. Pharmacol. 458: 235-241 [2003]), glycogen synthase kinase-3β (GSK-3β) inhibitors (Trends Mol. Med. 8: 126-132, 2002; Trends Pharmacol. Sci. 24: 233-238 [2003]), memantine (Neuropharmacol. 38: 1253-1259 [1999]), mixed cyclin-dependent kinase-GSK3β inhibitors (Oncogene, 20: 3786-3797 [2001]), COX-2 inhibitors (Neurobiol. Aging, 23: 327-334 [2002]) and propentofylline (Eur. J. Pharmacol. 458: 235-241 [2003]).

The present invention further provides compositions for treating otic disorders by administering a composition containing a p21^{Waf1/Cip/Sdi1} gene product protein (see below) inhibitor and/or inhibitors of cyclin dependent kinase-1 (CDK1), CDK2, CDK5 and CDK9, and inhibitors of cJAK and ASRK-1 including the following agents: olomoucine, roscovitine, purvalanol, kenpaullone, alsterpaullone, indirubins, flavopiridol, stauroporine and analogs and derivatives of the above compounds.

The Compounds of this invention can be incorporated into various types of otic/ophthalmic formulations for delivery to the ear (e.g., topically, intraotically, or via an implant). The Compounds are preferably incorporated into topical otic formulations for delivery to the ear. The Compounds may be combined with otically acceptable preservatives, surfactants, viscosity enhancers, penetration enhancers, buffers, sodium chloride, and water to form an aqueous, sterile ophthalmic suspension or solution. Otic solution formulations may be prepared by dissolving a Compound in a physiologically acceptable isotonic aqueous buffer. Further, the otic solution may include an otically surfactant to assist in dissolving the Compound. Furthermore, the otic solution may contain an agent to increase viscosity, such as, hydroxymethylcellulose, hydroxyethylcellulose, hydroxypropylmethylcellulose, methylcellulose, polyvinylpyrrolidone, or the like, to improve the retention of the formulation in the ear canal or inside the other compartments of the ear. Gelling agents can also be used, including, but not limited to, gellan and xanthan gum. In order to prepare sterile otic ointment formulations, the active ingredient is combined with a preservative in an appropriate vehicle, such as, mineral oil, liquid lanolin, or white petrolatum. Sterile otic gel formulations may be prepared by suspending the Compound in a hydrophilic base prepared from the combination of, for example, carbopol-974, or the like, according to the published formulations for analogous otic preparations; preservatives and tonicity agents can be incorporated.

The Compounds are preferably formulated as topical otic suspensions or solutions, with a pH of about 4 to 8. The establishment of a specific dosage regimen for each individual is left to the discretion of the clinicians. The Compounds will normally be contained in these formulations in an amount 0.01% to 5% by weight, but preferably in an amount of 0.05% to 2% and most preferably in an amount 0.1 to 1.0% by weight. The dosage form may be a solution, suspension microemulsion. Thus, for topical presentation 1 to 2 drops of these formulations would be delivered to the ear 1 to 4 times per day according to the discretion of a skilled clinician.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Formulation of Tanis Gene (TG) Inhibitor or TG Product Protein Inhibitor or p21^{Waf1/Cip/Sdi1} Gene Inhibitor or Inhibitor of p21^{Waf1/Cip/Sdi1} Gene Product Protein for Otic Use:

1% suspension or solution of Tanis gene inhibitor (TGI) or inhibitor of Tanis gene product protein (TGPPI) or p21^{Waf1/Cip/Sdi1} gene inhibitor (p21GI) or inhibitor of p21G is product protein (p21GPPI) for topical otic use:

| Description | Conc. | Units | Purpose |
|---|---|---|---|
| TGI or TGPPI or | 1% | W/V % | active ingredient |
| p21GI or p21GPPI | | | |
| hydroxypropyl | 0.5% | W/V % | viscosity modifier |
| methylcellulose | | | (2910) (E4M), USP |
| dibasic sodium phosphate | 0.2% | W/V % | buffering agent |
| (anhydrous), usp | | | |
| sodium chloride, usp | 0.75% | W/V % | tonicity agent |
| disodium edta | 0.01% | W/V % | chelating agent |
| (edetate disodium), usp | | | |
| polysorbate 80, nf | 0.05% | W/V % | wetting agent |
| benzalkonium chloride, nf | 0.01% | W/V % | preservative |
| sodium hydroxide, nf | q.s. pH | W/V % | pH adjust |
| hydrochloric acid, nf | q.s. pH | W/V % | pH adjust |
| purified water, usp | q.s. 100% | W/V % | vehicle |

In similar other examples, TGI or TGPPI or p21GI or p21GPPI will be substituted by agents that sequester or degrade the above amyloids and/or gene products of Tanis and p21^{Waf1/Cip/Sdi1}, or agents that prevent the toxic effects of the latter and/or amyloids.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

Methods to measure the potency or efficacy of agents that can inhibit the secretion of SAA from cultured cells involve the use of an enzyme-linked immunosorbant assay (ELISA) for human SAA as described by Yamada et al. (2000) and using human peripheral monocytes and monocytic leukaemic cell-line THP-1. In addition, methods to determine the potency and efficacy of agents to inhibit gene expression of p21^{Waf1/Cip/Sdi1} can be studies using standard methods described by Chang et al. (2000).

References

The following references, and the bibliography cited within these, to the extent that they provide exemplary procedural or other details supplementary to those set forth herein, are specifically incorporated herein by reference.

United States Patents

Other Publications
Abler et al., RES. COMMUN. MOL. PATH. & PHARM. 92:177-189 (1996).
Ambati, J. et al., Age-related macular degeneration: etiology, pathogenesis, and therapeuic strategies. Surv. Ophthalmol. 48: 257-293 (2003).
Asrani et al., INV. OPHTHALM. VIS. SCI. 38(13):2702-2710 (1997).
Asrani and Zeimer, BR. J. OPHTHALM. 79(8):776-780 (1995).
Bressler et al., SUR. OPHTHALM. 32:375-413 (1988).
Brown, M., Gene therapy success for Alzheimer 's? DRUG DISCOV. TODAY 8:474-475 (2003).
Caricasole, A. et al., The Wnt pathway, cell-cylce activation and β-amyloid; novel therapeutic startegies in Alzheimer 's disease? TRENDS PHARMACOL. SCI. 24:233-238 (2003).
Chabry J. et al., In vivo and in vitro neurotoxicity of the human prion protein (PrP) fragment P118-135 independently of the PrP expression, J. NEUROSCI. 23:462-469 (2003).
Chang, B-D. et al., Effects of p21^{Waf1/Cip/Sdi1} on cellular gene expression: implications for carcinogensis, senescence, and age-related diseases, PROC. NAT. ACAD. SCI, USA 97:4291-4296 (2000).
Ciullaetal., SUR. OPHTHALM. 43:134-146 (1988).
Curcio et al., INV. OPHTHALM. VIS. SCI. 37:1236-1249 (1996).
Fakforovich et al., NATURE 347:83-86 (1990).
Ge-Zhi et al., TRANS. AM. OPHTHALM. SOC. 94:411-430 (1996).
Gragoudas et al., INV. OPHTHALM. VIS. SCI. 38(4)S17 (1997).
Hock, C. et al., Treatment with the selective muscarinic m1 agonist talsaclidine decreases cerebrospinalfluid levels of Aβ₄₂ in patients with Alzheimer's disease, AMYLOID: J. PROT. FOLD. DISORD. 10:1-6 (2003).
Husain et al., OPHTHALM. 104(8):242-250 (1997).
Janus, C. et al., Aβ peptide immunizaion reduces behavioural impairment and plaques in a model of Alzheimer's disease, NATURE 408:979-982 (2000).
Jen, L S. et al., Alzheimer's peptide kills cells of retina in vivo, NATURE 392:140-141 (1998).
Jensen L E and Whitehead A S, BIOCHEM. J. 334:489-503 (1998).
Johan, K. et al., Acceleration of amyloid protein A amyloidosis by amyloid-like synthetic fibrils, PROC. NAT. ACAD. SCI USA 95:2558-268 (1997).
Kane, M. D. et al., Inhibitors of V-type ATPases, bafilomycin A1 and concanamycin A, protect against β-amyloid-mediated effects on 3-(4,5-dimethylthiazol-2-yl)-2,5-diphenyltetrazolium bromide (MTT) reduction, J. NEUROCHEM. 72:1939-1947 (1999).
Kindy, M. S et al., Apolipoprotein serum amyloid A in Alzheimer's disease, J. ALZHEIMER'S DISEASE 1:155-167 (1999).
Koriyama, Y. et al., Propentofylline protects β-amyloid protein-induced apoptosis in cultured rat hippocampal neurons EUR. J. PHARMACOL. 458:235-241 (2003).
Kumon, Y., Hosokawa, T., Suehiro, T., Ideda, Y., Sipe, J. D., and Hashimoto, K., Acute-phase, but not constitutive serum amyloid A (SAA) is chemotactic for cultured human aortic smooth muscle cells, AMYLOID 9:237-241 (2002a).
Kumon, Y., Suehiro, T., Faulkes, D. J., Hosakawa, T., Ideda, Y., Woo, P., Sipe, J. D., and Hashimoto, K., Transcriptional regulation of Serum Amyloid A1 gene expression in human aortic smooth muscle cells involves CCAAT/enhancer binding proteins (C/EBP) and is distinct from HepG2cells, SCAND. J. IMMUNOL. 56:504-511 (2002b).
Kumon, Y., Suehiro, T., Hashimoto, K., and Sipe, J. D., Dexamethasone, but not IL-1 alone, upregulates acute-phase serum amyloid A gene expression and production by cultured human aortic smooth muscle cells, SCAND J. IMMUNOL. 53:7-12 (2001).
Lambert, M. P. et al., Diffusible, nonfibrillar ligands derived form Aβ₁₋₄₂ are potent central nervous system neurotoxins, PROC. NAT. ACAD. SCI. USA 95:6448-6453 (1998).
Lanz, T. A. et al., The γ-secretase inhibitor N-(N-(3,5-difluorophenacetyl)-L-alanyl]-S-phenylglycine t-butyl ester reduces Aβ levels in vivo in plasma and cerebrospinal fluid in young (plaque-free) and aged (plaque-bearing) Tg2576 mice J. PHARMACOL EXPT. THER. 305:864-871 (2003).
LaVail et al., PROC. NAT'L ACAD. SCI. 89:11249-11253 (1992).
Liang, J. S., Sloane, J. A., Wells, J. M., Abraham, C. R., Fine, R. E., and Sipe, J. D., Evidence for local production of acute phase response apolipoprotein serum amyloid A in Alzheimer's disease brain, NEUROSCI. LETT. 225:73-76 (1997).
Lin et al., CURR. EYE RES. 13(7):513-522 (1994).
Liu, Y and Schubert, D., Cytotoxic amyloid peptodes inhibit cellular 3-(4,5-dimethylthiazol-2-yl)-2,5-diphenyltetrazolium bromide (MTT) reduction by enhancing MTT formazan exocytosis, J. NEUROCHEM. 69:2285-2293 (1997).
Marks, N. and Berg, M. J., APP processing enzymes (secretases) as therapeutic targets: insights from the use of transgenics (Tgs) and transfected cells, NEUROCHEM. RES. 28:1049-1062 (2003).
Matsuoka, Y. et al., Novel therapeutic approach for the treatment of Alzheimer's disease by peropheral administration of agents with an affinity for β-amyloid, J. NEUROSCI. 23:29-33 (2003).
Miida T., Yamada, T., Yamadera, T., Ozaki, K., Inano, K., Okada, M., Serum amyloid A protein generates pre-beta 1 high-density lipoprotein from alpha-migrating high-density lipoprotein, BIOCHEM. 38(51):16958-16962 (1999).
Morgan, D et al., Aβ peptide vaccination prevents memory loss in an animal model of Alzheimer's disease, NATURE 408:982-985 (2000).
Naash et al., INV. OPHTHALM. VIS. SCI. 37:775-782 (1996).
Nakagami, Y and Oda, T., Glutamate exacerbates amyloid β1-42-induced impairment of long-term potentiation in rat hippocampal slices, JPN. J. PHARMACOL. 88:223-226 (2002a).
Nakagami, Y. et al., A novel-sheet-breaker, RS-0406, reverses β-amyloid-induced cytotoxicity and impairment of long-term potentiation in vitro, BR. J. PHARMACOL. 137:676-682 (2002b).
Noell et al., INVEST. OPHTHALM. VIS. SCI. 5:450-472 (1966).
O'Hara, R., Murphy, E. P., Whitehead, A. S., FitzGerald, O., and Bresnihan, B., Acute-phase serum amyloid A production by rheumatoid arthritis synovial tissue, ARTHRITIS RES. 2:142-144 (2000).
Pike, C. J. et al., Neurodegeneration induced by β-amyloidpeptides in vitro: the role of peptide assembly state, J. NEUROSCI. 13:1676-1687 (1993).
Schenk, D. et al., Immunization with amyloid-β attenuates Alzheimer's-diesease-like pathology in the PDAPP mouse, NATURE 400:173-177 (1999).
Sickenberg et al., INV. OPHTHALM. VIS. SCI. 38(4):S92 (1997).
Taylor et al., ARCH. OPHTHALM. 110:99-104 (1992).
Thomas et al., INV. OPHTHALM. VIS. SCI. 39(4):S242 (1998).
Thorn, C. F. and Whitehead, A. S., Differential glucocorticoid enhancement of the cytokine-driven transcriptional activation of the human actue phase serum amyloid A genes, SAA1 and SAA, J. IMMUNOL. 169:399-406 (2002).
Uhlar, C. M., and Whitehead, A. S., Serum amyloid A, the major vertebrate acute-phase reactant, EUR. J. BIOCHEM. 265:501-523 (1999).
Urieli-Shoval, S., Cohen, P., Eisenberg, S., and Matzner, Y., Widespread expression of serum amyloid A in histologically normal human tissue. Predominant localization to the epithelium, J. HISTOCHEM. CYTOCHEM. 46:1377-1384 (1998).
Walder et al., Tanis: A link between type 2 diabetes and inflammation? DIABETES 51:1859-1866 (2002).
Xia, W., Relationship between presenilinase and γ-secretase, DRUG NEWS PERSPECT. 16:69-73 (2003).
Yamada et al., Serum amyloid A secretion from monocytic leukaemia cell line THP-1 and cultured human peropheral monocytes, SCAND. J. IMMUNOL. 52:7-12 (2000).
Yamazaki et al., BIOCHEMICAL AND BIOPHYSICAL RES. COMM., 290:1114-1122 (2002).
Young, SUR. OPHTHALM. 32:252-269 (1988).
Zhang, L. et al., α-Lipoic acid protects rat cortical neurons against cell death induced by amyloid and hydrogen peroxide through the Akt signaling pathway, NEUROSCI. LETT. 312:125-128 (2001).

1. A method for treating otic disorders, said method comprising administering to a patient in need thereof a therapeutically effective amount of a composition comprising an agent that interacts with a gene encoding a serum amyloid A (SAA) receptor (SEQ ID NO:12), wherein said interaction decreases the expression of SAA (SEQ ID NO: 1 or SEQ ID NO:3).

2. A method for treating otic disorders, said method comprising administering to a patient in need thereof a therapeutically effective amount of a composition comprising a Tanis antagonist.

3. The method of claim 2, wherein said agent is a peroxisome proliferator-activated receptor α (PPARα) agonists, tachykinin peptides and their non-peptide analogs or α-lipoic acid.

4. The method of claim 3, wherein the agent is fenofibrate, Wy-14643, (4-chloro-6-(2,3-xylidino)-2-pryrimidinylthiol)-acetic acid), ciprofibrate, 2-bromohexadecanoic acid, bezafibrate and ciglitizone, bafilomycin or concanamycin.

5. A method for treating otic disorders, said method comprising administering to a patient in need thereof a therapeutically effective amount of a composition comprising a p21 antagonist.

6. A method for treating otic disorders, said method comprising administering to a patient in need thereof, a therapeutically effective amount of an agent that down-regulates expression a tanis gene (SEQ ID NO:14) or p21^{Waf1/Cip1/Sdi1} gene (SEQ ID NO: 16).

7. A pharmaceutical composition for use in treating otic disorders comprising a therapeutically effective amount of a Tanis antagonist and a pharmaceutical carrier.


SEQUENCE LISTING (17 sequences)

SEQ ID NO: 1 - DNA, 369 bp, homo sapiens
atgaagcttctcacgggcctggttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgtctgggctgcagaagcgatcagcgatgccagagagaatatccagagattctttggccatggtgcggaggactcgctggctgatcaggctgccaatgaatggggcaggagtggcaaagaccccaatcacttccgacctgctggcctgcctgagaaatactga

SEQ ID NO: 2 - protein, 122 aa, homo sapiens
MKLLTGLVFCSLVLGVSSRSFFSFLGEAFDGARDMWRAYSDMREANYIGSDKYFHARGNYDAAKRGPGGVWAAEAISDARENIQRFFGHGAEDSLADQAANEWGRSGKDPNHFRPAGLPEKY

SEQ ID NO: 3 - DNA, 570 bp, homo sapiens
agggacccgcagctcagctacagcacagatcagcaccatgaagcttctcacgggcctggttttctgctccttggtcctgagtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggccgcagaagtgatcagcaatgccagagagaatatccagagactcacaggccatggtgcggaggactcgctggccgatcaggctgccaataaatggggcaggagtggcagagaccccaatcacttccgacctgctggcctgcctgagaaatactgagcttcctcttcactctgctctcaggagacctggctatgaggccctcggggcagggatacaaagttagtgaggtctatgtccagagaagctgagatatggcatataataggcatctaataaatgcttaagaggtcaaaaaaaaaaaaaaaaaaaaaaaaaaaaaa

SEQ ID NO: 4 - protein, 122 aa, homo sapiens
MKLLTGLVFCSLVLSVSSRSFFSFLGEAFDGARDMWRAYSDMREANYIGSDKYFHARGNYDAAKRGPGGAWAAEVISNARENIQRLTGHGAEDSLADQAANKWGRSGRDPNHFRPAGLPEKY

SEQ ID NO: 5 - DNA, 4286 bp, homo sapiens
gatggttgacaactcccctcctcttccccctcttctactgtctactcctgggaccaagtgagccacgccagctcagatactacactgaccacagggaatcccaccttttccaaggaatggaagttgtgtagggaatattcaaatgttgcttagcattgccttagataagaaccaaagggacagggaaatcctctgacagctatctgccttataactttcattttactgtgcctaaaatatgctcagaacccagaaagaggcataattcctaattttggcaggctctaatctaaaataatgattctcaaacatggtgtgacttttgtctatttgctttatcctgggtcactgctcctcttctgtcagatactgggattccaatgagacaaatggaaatggagacgtagaccctctgaccttctatcttttatctatacacatacacctgtgtgtgtgtgtgtgtgtgtgtgtgtgcgtgtgtaaaaccgagtgggtttttttcttggaatgaaagaatggactaacattacaaaaaataaaaacttgaaacagaatgtgtattatccttggttgtgtttccttggccctgcagcaggatgaagctctccactggcatcattttctgctccctggtcctgggtgtcagcagccaaggatggttaacattcctcaaggcagctggccaaggtgaggtccacaggatagggggcaggaggctgcttctggctgcccccaggatgcagctgagcagaggccacatccccactgggcaaaggtgctagtgatgccacagatggatagagaaggggcatggtttttcataagcgtggttcctcatgcttttctggacagctttgacactcttctatgaggatcctccagccgaggtcgcataaggtgtgagctgcctcttttcagcaggaccatgagagagatgtggagttgaggggtgcatgttcccataataccggtggggctctactgccccctagtgggaaatctgggacagttcatgtctatgtctcctgggaagccaggaagcaggtggatcaaaagtgtgaggcgagtccatggggaagctgaacggagccaaccgtccccataaaaacaaccaagcttagctgagattttaatacgtactaggcactgtttaaatgtactaatgaattggtttccatcatttagtcctatgatgcaagcagcattatcccttaacagagaagctaacacacacacacacacacacacactaacacacacacacacacacacacacacacaaaccccaagatacgtaaagaagttccaaagcagagcaggattaacccaggcagtcttgctctgcagaacttgctcttaatcaaggtactctgctgctttcaaaacaagagtttcggatttgtgaacacatagctcatcctttatctaagaaatggcaaataggatgtggtgcctttggaaggtaagtctagctccacttatcccagtaaaacctacagtgaattaccttgatggtggttctactggggcttatatatggccaggaaactgctagcaagagaaatataccccgagggctgggcacagtggctcacacctgtaatcccagcactttgggaggctgaggtgggcagatcacctgaggtcaagagttcgagaccagcctggccaacatggcgaaatcctgtctctactaaaaatacagaaattagccgggtgtggtggcatgcgcctataatcccagcctctcgggaggctgagggagaagaattgcttgaactcaggaggcagaggttgcagtgagctgtgatcacaccactgcactccagcctaggagacagagcaagactccatctagagagacagagagagagagagagggagaaatataccccactagccataataaagtggcaaaattttgttttcagaatgcagtattttaaatttcaggtattattatttttctgagtctctgaaaaatggttttaaggatttgcttttaatcctatttacatgttcacacactcaactacaaatatctttcattccttaggttaatatttttcaaagggttgttctgggaccacttgcgtgagaatcacctggattctgggatgctttgtgaaatgaaatgaagattcccgggtccataccctaccccctgcccccaacagccacagtctcttgggacagagcctagaaatcttgcctttgctaagcacctcggtagatttttatgcacagcaaaggttgagaaccactacctcttgttttgctgctgaaagtgataaaatgtgccaggaattttggaagtacttattaagccaatctgaacatcaaggagccatttaagtcagtaactcagaggaataagtagagtaaaaatgtcataaactctcaataaaagcaatcaatttaacaccaggagtaataaatgcataaaatgaagatgagttatctaatagagaaattatataaaccatgattataactctatatttgagttcccccttttccgtaatcagttaattttctaaaaaatcttcgtcacttaattctagcttgatcagatcccttcagtccgtaactccctgctcctcatcttagtttagcccttcttttttcttatgccacctttcctaaggaccagagaagtgaaatgataatatattggccacctacaatgttctagacatcatacatgtattttctctgctcttctgcataatcactgtgaggcaggcaatactcctccatttcattggggaggacattgaggttctgaactagtgggtcagttgtcctttttctgaatttgattacccagtagtataaagctttcttaggtaactcacctttatcacttgctgactgaattctgacagatgtcagtttctaattatagcctggacattcagatgtattcaggaccaagttgtcctcactctacctacaggcatgaatttctctcattgactaggttaggagcgccatatgtctgcagcctccctcagaatcccctgtgttctcacaccagggaactgagggttccctgggtccttccaggtagaagttcattgtacaatgaaacatcccttaaggaccatttcatctcttctttaggtgcatcacacatggttaaaacaaagtaataacagaacttagaatggaatcaaacagaatgaaacttacaccaagtacaattctcattacattaacccagagaagtgaaaagtagaagaatatttatttcaagccaatataatttccaagggctttgttgaaggctgaaatcttcgggaggaaagtagtgagaagaaaactgttcattcctctattttcccagtatataattgttttgatcattttctttcctttccagggactaaagacatgtggaaagcctactctgacatgaaagaagccaattacaaaaaattcagacaaatacttccatgcttgggggaactatgatgctgtacaaagggggcttggggctgtctgggctacagaagtgatcaggtaatgcacattcctgatgttgccaggaatgagtgagcagagcttgactgccttggacagtcaggagagaggtaagctccttgcagagaagttagaggctgcagcccctcctcctcttgccctctctctgcctgtgtgcttagtgcgagggtctgagtggatggtagaagtgagtgattcctcaccctccctctctgggtgctgttcatccagcctaggggtgcccagcctggctgagtggggcagtgcccaggcagggtcattgttttcacccctccttccttggccttcctgggcttctcccagagtcctcccttggaaagcagagaatgggaaggtgggctgttgctcactggcctggtgattaatctccttgcttgcctggactacagcgatgccagagagaacgtccagagactcacaggagaccatgcagaggattcgctggctggccaggctaccaacaaatggggccagagtggcaaagaccccaatcacttccgacctgctggcctgccagagaaatactgagcttccttttcaatctgctctcaggagacctggctgtgagcccctgagggcagggacatttgttgacctacagttactgaattctatatccctagtacttgatatagaacacataaaaatgcttaataaatgcttgtgaaatccagtttgttattggaatctggaagcagaatatgacagtcttcctgggatcatgggcctgtttagtaccatagggatgaccaataaac

SEQ ID NO: 6 - DNA, 193 bp, homo sapiens
gttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtctgggct

SEQ ID NO: 7 - protein, 64 aa, homo sapiens
VFCSLVLGVSSRSFFSFLGEAFDGARDMWRAYSDMREANYIGSDKYFHARGNYDAAKRGPGGLG

SEQ ID NO: 8 - DNA, 369 bp, homo sapiens
atgaagcttctcacgggcctggttttctgctccttggtcctgagtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggccgcagaagtgatcagcaatgccagagagaatatccagagactcacaggccatggtgcggaggactcgctggccgatcaggctgccaataaatggggcaggagtggcagagaccccaatcacttccgacctgctggcctgcctgagaaatactga

SEQ ID NO: 9 - protein, 122 aa, homo sapiens; identical to SEQ ID NO: 4

SEQ ID NO: 10 - DNA, 369 bp, homo sapiens
atgaagcttctcacgggcctggttttctgctccttggtcctgagtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggccgcagaagtgatcagcaatgccagagagaatatccagagactcacaggccgtggtgcggaggactcgctggccgatcaggctgccaataaatggggcaggagtggcagagaccccaatcacttccgacctgctggcctgcctgagaaatactga

SEQ ID NO: 11 - protein, 122 aa, homo sapiens
MKLLTGLVFCSLVLSVSSRSFFSFLGEAFDGARDMWRAYSDMREANYIGSDKYFHARGNYDAAKRGPGGAWAAEVISNARENIQRLTGRGAEDSLADQAANKWGRSGRDPNHFRPAGLPEKY

SEQ ID NO: 12 - DNA, 10001 bp, homo sapiens
gggtggatcacgaggtcaggagatcgagaccatcttggctaacatggtgaaaccccgtctctactaaaaatacaaaaaaattagccgggcgtcatggtgggcgcctgtagtcccagctactcgggaggctgaggcaggagaatggtgtgaacccgggaggcagaacttgcagtgagcctagatcgcgccactgcactccagcctgggggacaaaacgagactctgtctcaaaaaaaaaaaaaaaaattcccacattagagttggggaaatgggcagtcctggtggaagttagggaacagatctgggacacgttatagccagctggactacaggaggccataagctcaattcttccttgactctgaaaccttccactggtcctaatgcctagtaattccaggcctttcccagttgtgccaggcttggaggtgaacacatctatgtgccaagaaggaaaggtatgccaagcaggggcttaagtcatccttatcctcagtctgtctatgagtggtatgtacccctgttccccttgcaagatctgctgggcttaggtctcctggctgtgagttccccatacctgggcataaatgtagtgagcctgagctcccaaataaggttgggggctccagagaggtggagagccctgtgtctgggaagtgtgcccacccagcaggtctgaccaggaagatacactgctagggttatggaaaaagactatgtgtcaaggtctcttgattctccatctaggcagagaatcatctttaattaatgggaaactggaaggcaaattacttggacctgaaattactttttgtttattgaaccactgtgttgtaaatcacatctctctgaaggcaagagaaatcagggagttacaaaatgtttaggagaactaaacaggactccctgttttgctaactaatcagattgagacaggctctctggtaaatctacaaatttgatgttgttcaaccataagcagtaaatttcctatgctggattttcctgacaatgaatgtaaaaggaaaaggagtctttttgacaaaatattttattgttcatctaaactgaaaaacttctctatttttcaaaattgctatacgtgtttaaagatgtagatatttgaatagcctaactggtacagaaggtttaatgatgattcctaagacatacctataaattacttgaaattgaaacgaaatttaagaagaattattggaattttccccttctcaaatgagttcttagtttcataaatactatacaagtccataagagatttggggttttgagatgtctttttttttttttttttttcagacggagtttcactgttgttgcctaggctggagtgcaatggcgtgacctcagctcactacaacctccacctcccaggttcaagcgattttcctgcctcagcctcccaagtagctgggattacagggacctgccacaacgccaagctaatgttttgtatttttagtagagatggggttcaccatgttggccaggcttgtctggaactcctgacctcaggtgatccacccgcctataatttattactcccttttgcaaatgtttgaaaaggaataaagtgcaatatttttaaacagaatgcagagttctgttgtcctttggcaataccagtttcagactctgagagtggctcttgctgttgccgacagtgggctgatgaccaaatcccaacatgcccccgctgcgagtccttcataacctgattcagtcatcacttagaggccagcaggcttcagggaggcgtgagcctcagccaacaacctataggggaagagacgcagaactcaatgcagacaggtttggattctggtgcctagagaatgcaacttggaaactctgagccaggagaaaagggttctctctccatgagagagtgtgggctttgtgagaagcgacacacagcaaacacaattaagagtccacccctcagcggggcgcaggggctcacgcctgtaatcccagcactttgggaggccgaggcgggtggatcacgaggtcaggagatcaagaccatcctggctaacacagtgaaaccctgtctctactaaaaatacaaaaaaattagccgggcgtggtggcgggcgcctgtggtcccagctactcgggaggctgaggcaggagaatggtgtgaacccgggaggtggagcttgcagtgagccgagatcgcgccactgcactccagcctgggcgacagagcgagactccatctcaaaaaaaaaaagaaaaagaaaaagaaaaagagtccgcccctgaattaaatagttggtccttttgtgttcctggtgattcacttgctaagtggaagaaacaggagggaatcttttctcctgccctcctggtaatccatagcccatggcctggctttacttctgtaaagtggcaggagaccttttgacagctgagccatttcttattttatttattttaataagagatggtaggaatgagcaatgatattagtacctggggactgttgttcttaaggagaaacaatcttagaatgattagtgataccccttgctttctcttttctttcattatactttttgtacacatatttttcccatttatttattggaatcttactgatttattataagtataagctttatgtctacacatgtataatcatttttccccaagtataagtctctttttcatggaggcacagcctagacctggttagccgccatctcccctcattgtatgcccaatatctattgtagtatctgctgcatagaaggcactcgatgcgtgaatggataatgactgatgatgaatcaataaataaatggacatgtcattgtaaaaaattctaaaaatctagaataacacaagctgttggcactacctagaaacacagatgtaaaacttcctaggttgtgtttcaccatgggaacatgtctttgaacaaaaatgggatcatattctattgcactctttcccttaagagatacttctccaggtcattaagtgctcttccacaatatcagtatatggcagaggcaaggtcataccaggtctgtctgaaaccagggcttggctcttaacttgcagccatactgcctccaagtctaggtggctgggttttaggatctgtaatgggaactcagtgtcacaacctctactgggaaggtattctggtgttgcataacaggactttctgttagagataaccatggcaaaatggaatagagacaaagttcaggtttctgctgccaggagctgagattgctgtgaccaatggcattctcccaaaccaaataatccaacctggaattaccataaaccactcctcatcttttcaaggggtgtccaagttcccagaaaagaacatttgttaagggatggaggcaaggaggtggagaagaaagagcactggccaaggtatcatgagtgtcctgggttctggtccttgaataagccatttatcttctctgcagcttctccatctgataggagtttggaggcagagttttttcttaatgagcaaaagacagtcgtgcctaggagatgtggtgtacatgttagaaagaagggactggctgtgactctataaaagatgaattcatacaaaaacaaattaccctttcccagggagaaagtttggatccagtaattagagatctcaaaaagtagaagacctgccctgtgaggcctgtggcctccaagtttgaatgctgtgtgtcagctttaaaaactagtttcttgctgataaatgtttcatattaagcatgtgttgagagtactccttgcctaccttcactagccactgtttccttcccctcctcccttgtcccttcattctctccagaactttctgctaacttccattctcttcaggacttcagcatggttgggagaagatcagaaaggcatcctcactgtttttattttagtccacttgacctttggggagtagttccactggctcataagtatcagccccccatagcacagcaccccacactgagcccggaagcaataaagaatcccaatctgctgtcactaaccagcacgctcaactgccatgccctttactcttctcatctccctgctttcacgtcacaccaactaatttctctatgagtcagcctcaactctcccaacactctgcccacccttcttctactaccttccagtgagctcctcgaaagaagggtctgcggtgaggatgcccctttatctctgcctatttccttcccattacaaaaacttgaaacctgcctttcccatgttgatttcactttattctcatctttacccatggggtatgcctcctgcaattcctcctagacaatagaatgagaaagaggggtcctcgtcctctttgctttccatgaccatttctccattcttcacctctgtgatgtgtcctctttgaagtccctgataaattcattaccaccttctctccagtcttactaatgttatctgcacaagtgatttccaaacaggaagattttcaaacactgattcctgaagatcacccccaactcgctgaactgagaccaagacctccaagattatggcttaggaatctgcattttttttttttttttgagacaagagtctcgctctgttgccaggctagagtgcaatggtggaatcatagctcattgtaacctcaaactcctgggctcaagtgatcttcctgcctcagcctcccaagtagtgaggacaacaggagtgtgccaccatgcccagctaattgttaattttttgtagaaatggagtctcactatgttgctcgggctggtctcaaactcctgaccttaacccatcctccgcctccgcccccaaaagtgttgggattacaggtgtgagccaccgtgcccagcctagaaatacccactagaagcttctgtgtagacaatctgcttagtgatgtttggagacaaagtacctctttattgtattcattgacaaaactctccagtcctctcccatcttcatggaaaattttcacagttcatttacggccctctttccaacacattcactgccaatactcttattgacaataactgtattgttgaaccttccagtatcctgcattcccggatcaaggccccctcaaagccctgatatgcaaatatctgggaaaagaatgttccagaggaaaggaacagctaatccgaggcccctagggtaagatgtgcctgggggtttggagaccagtgtggccagagcaaaatgagcaggaggagagaattggatgatgaggtacgagaggaaggagttaggacagtttgagtaaagtttgaaaaccattataagggctttgacttcaactatgagtggaagtggaatcctccggagagttttgaatggagagtgatagaagttgtcttgtgttgtaacagtctggctgctatactgaaaagagactagttggcggcaaagggggaaatgtggaagccagttaagaagccatcataacccagaaggtgatgcctaataacatctctctgggagcagcggagagatgataagggtttgccttctgaatatgttttttgacaattaatgtaaacatttcaagtaggctgagattttattgcatattaacaatgtccatgttcactcgcggcagccgcccccttctgcgcggtcatgccgagccagcacctgggcctggaactgggccgcagcccccagcttcacccaccacctccctaccatggacccctgcaaagtgaacgagcttcgggcctttgtgaaaatgtgtaagcaggatccgagcgttctgcacaccgaggaaatgcgcttcctgagagagtgggtggagagcatgggaggtaaagtaccacctgctactcagaaggctaaatcagaagaaaataccaaggaagaaaaacctgatagtaagaaggtggaggaagacttaaaggcagacgaaccatcaactgaggaaagtgatctagaaattgataaagaaggtgtgattgaaccagacactgatgctcctcaagaaatgggagatgaaaatgtggagataacggaggagatgatggatcaggcaaatgataaaaaagtggctgctattgaagtcctaaatgatggtgaactccagaaagccattgacttattcacagatgccatcaagctgaatcctcgcttggccattttgtatgcaaagagggccagtgtcttcgtcaaattacagaagccaaatgctgccatccaagactgtgacagagccattgaaataaatcctgattcagctcagccttacaagtggcgggggaaagcacacagacttctaggccactgggaagaagcagcccatgatcttgcctttgcctgtaaattggattatgatgaagatgctagtgcaatgctgaaagaagttcaacctagggcacagaaaattgcagaacattggagaaagtatgagcgaaaacatgaagagcgagagatcaaagaaagaatagaacgagttaagaaggctcaagaagagcaggagagagcccagagggaggaagaagccagacgacagtcaggagctcactatggcccttttccaggtggctttcctggtggaatgcctggtaattttcccggaggaatgcctggaatgggaggggacatgcctggaatggccggaatgcctggactcaatgaaattcttagtgatccagaggctcttgcagccatgcaggatccagaagttatggtggccttccaggatgtggctcagaacccagcaaatatgtcaaaataccagagcaacccaaaggttatgaatctcatcagtaaattgtcagccaaatttggaggtcaagcataatgcccttctgataaataaagccctgctgaaggaaaagcaacctagatcaccttatggatgtcgcaataatacaaaccaacgtacctctgaccttctcatcaagagagctggggtgctttgaagataatccctacccctctcccccaaatgcagctgaagcattttacagtggtttgccattagggtattcattcagataatgttttcctactaggaattacaaactttaaacactttttaaatcttcaaatatttaaaacaaatttaaagggtctgttaattcttatatttttctttactaatcattgtggatttttccttaaattattgggcagggaatatacttatttatggaagattactgctctaatttgagtgaaataaaagttattagtgcgaggcaaacataaaaaaaaaaagtccatgttcatctctaaatgacatcattgttccaaagcttttccattcttcttaaccttccacctgtcaatctataggagatgacttctcctacttcactcatgcattgactccttcaatcaataaaagtgactaagaacctgctacaggtgaggtgctgtgtttggtgttaaagtgacaacagttatctgtcaataagcctgacaaggttcctatccctgtgttttgtgcactctgggtcaaactcagaaatgcaaacaggtggagagcgatgagttctatgactggtaaagaaaagggcctgctggtttccctcaggatctctgtccttcatctcaaaatgcatcttccttgttatcgttcctctccttcctgtctcagaggaagacctgctcctgctacactctgggcaaccttgtccccgtggccctgtggccccttggttgttgaagtctatgttatgccctatcttttaccctcagtcactctctctgttaacattctccctgtgccctgtaaccctccctcatctttaaataaatcctcctcctttgaccttcgcatgtattcagtcatgcaactcaacaagcatttattgcacagtgatattcaatttgccacttgctaaaagtctgaaccttggcagctgaatgtgatcagaaaaaaagcacgactgctatgactagtctcactttaaattcatggtcgttgaccaagagctaccatacaatccactacctttctcaagttcagtcacattcttcctttcctagatgtctgctttctacttctcttctcttctgaaacttcccacaactcctcgttcattctcttctcagttgacaactttgcttcctatttcactgaaaaatagaagcaatcagatatgaacttctggctgggcatggtagctcatgcctataatctcagcactttgggaggccaaggcaggaggactgcaggttaggaatttgagaccagcctgggcaacatggtgaaactcccactgtactaaaaattttaaaaattactcaaacatattggcaaacaactgcagtcccagctacttgggaggttgagatgcaaggatcacttaaacctgggaggctgaggctgcagtgagccatgattgcaccactgcactccagctcaggcaacagagcaagaccctgtcttgagaggagaggagaagagaggaggggaggggagggcaggggaggggaggggaggggaagggagaggggaggggagaggggaggagagaggggaggggaggggaggggaggggaggggaggagaggaggatcaggtgaggagtatgccaaggagtgtttttaagacttactgttttctctttcccaacaagattgtcatttcctttaaaaagtagttatcctgaggcctatattcatagcattctgaaagaaagaaaagaaaagaggaaagaaagagagaggaaggaaggaaggagaaagagagaggaaggaaggagaaagagagaggaaggaagggaggaagagaagaagggaggaagaaaagaaggaaggaaggagggagggagggaagggagggagggaaagaggaagaaaggagggaaagaaggaaggaagagagagaggaaggaaggaggaagagagaagaaggaaggaggaagacagagagggagtaaggaaggaaggaaggagaaagagagaggaaggaagaaatgaaggaaggaaggaaagaaagaaaaaataaaagagtgaaaacggactggagaagaagaaaccacagttgctgctatatccaccagcctctctgcatgtcctggcctcagccctgctgggctctggtactgaccacttccttccttcctaatttcctaattgactaggccagctgagcagggcttttctgtgctgaggaggtaaatctctggatatctagactgaggggtggaaggagccttccagggcacacatgagacatggcaggggtaggctgctagttttattttgttttcttttagacacagggtcttgctctgttaaccaggctggagtgcagtggcgtgattatagctcactgcagccttgacctcctgggtctcccacaatccttccgcttcagcctcttgagtagctgggactgcaggtgcacactaccacacccggtccatttatttttatatttcgtagagacaagatcttacagttttgcacagagtgatcttaaactcttgaccccaagtgatcctcctgccttggcctccaaaagcattgggattataggagtgagccactgtgctggacctagtctgtcagctttgaagctttagatatgaactcagagggacttcatttcagaggcatctgccatgtggcccagcagagcccatcctgaggaaatgactggtagagtcaggagctggcttcaaagctgccctcacttcacaccttccagcagcccaggtgccgccatcacggggctcccactctcaactccgcagcctcagccccctcaatgctgaggagcagagctggtctcctgccctgacagctgccaggcacatcttgttccctcaggttgcacaactgggataaatgacccgggatgaagaaaccactggcatccaggaacttgtcttagaccgttttgtaggggaaatgacctgcagggactttccccagggaccacatccagcttttcttcgctcccaagaaaccagcagggaaggctcagtataaatagcagccaccgctccctggcaggc

SEQ ID NO: 13 - DNA, 10001 bp, homo sapiens
gtctgccagggagaggtggctgctatttatagtgagccttgctggtctcttgggagggaagaaaagctggatgtggtccctggggaaagtccctgcaggtcatttcccctacaaactggtctaagacaagttcctggatgccggtggtttcttcatcccgggtcatttatcccagttgtgtaacctatgggaacaagagaggtttgctgtgccttggcaatggacagggtgctagatcagctctgctcctcagcattgggggaagtgcagctgcagagatgccagtgggagccccgtgatggcggcacctgggctgctggaaggtgtggagtgagggcagctcttcagccagctcctgactataccggtcatttcctcaggatgggccctgctgggccacatggcagatgaccctgactgaaatccctgtgagttcatgtctaaagctttaagctttaaaacggacagcctacccctgccacatctcatgtgtgccctggaagcctccttccacccctctggatgtcctgatatttctcagcacagaaaatctctgctccgctggcttagccaatttggaaatgctttttctaagttggctcctgagccaaggacaatgtagagagggggactttctgctgccccagcctagtcctggagccccaccttgggagaatgagagtgtggtgcgttaaataggcagcccagctggggacgtgcccagcatccaggcagggaagggtgggagagctcttggtctgctgtattatcacggaggggtgcagggggcatgcagatcactctctcatgagaacatcaacagggtcagattagctctgcagaggcttatggaggagcatggtggccagagatgggtcagtaccagagcccaggggggctgaggccaggacatgcagagaggctggtggacatagcagcaactctggtttcttcttctccagtccatgttcataccctgagggctaggcatttgtaataacaaacaaacaagcaatttagaaatgggccaggcatggtggcatgtgcctatagtcccagctacttgggaggccaaggcaggaggcctgcttgaacccagaaatttgaggccagcctgggcaacacagcaagattatcttaaaaaattttttttaatctctgagaaatgggtagggccaggaagtaaaggatggccaaatactccataagcagcaaatgcgtggctccaatgtgaacaatgatattatagactctgttctgagacctatgcattgacacctccacctcccccactacatcttgccaccttaaaaccactgagagtggtacctgctggaatgggtccacacacacagtcacacatattttaggcagggtagttgacatccccagggaaaaagagctcacagagagaggctgaatgtttccaactgggtagcagtaatagtacatcatgctgtacatggtacagcacagatcaggtgaaaataatagcacatcgtgattaaccagggcttattccagggagtcaagaagagtttcatatcagaaaaatctatctttgtaattcactataccagtaatcaaagaaaaggattgtacatttattttactagatgcagaaaatgaatttcataattgtcaacatctactgatgataaggaaaatgtataacaaaataaagagaccatttctgacttgagaaaggataaataccaatatgttatagcaacagttctcaaactgttttccagggaaccctaagaatccctccttagggaggctttgatctcaaaattatttttagaatagtgctaacacactattttcatgtttcagtctcattttctcatgagtacacacaatatgacaagttagttgatatgagtgtggatttccacatggtaactgacttttcagaagctaccacttgttgagtttggtataatatagaatagccacaattatctaaaaataccattaaaatacactcccccatttcaactatatatctgtgtgaggctgaattttcttcatatactccaacctaaataacatattaaaacaggttggatgatgaatcagataggaaaatccagctatgaaaaaaaaatcagacatgaaaaattttcaaaagggtaaaaccatagtactcttcttactttttttcttttggaagatggttatttttcataaaaatatattatttatgttaacatatagaagatggataattttttgaagaattgataaatgtttaaattttttctttctattatggtaaatactgatgaatagagtccccataaataaaagttctttggggtattcaataatttttaatagtgtaatgggatcctgagaccaaaaggtttgagaatcattgctctacagcaaacattatgtgtaattaagacacttcaggtgcattctcaagaagaccaataaagaggccacaatggcaggcgtggtggctcacacttgtaatccaagaacttagagaggacgaggcaggtggatcactggaggtcaggaattctcaaccagcctggccaacatggtgaaaccctgtctctactaaaagtacaaaaattagtcgggtgtagtggcaggtacctgtaatcccaagtacttggggggttgaggcaggagaatcacttgaagccgggaggtggaggctgcagtgagccgagatcgtgccactgcactccagcctgggcaacggagtgagacttcatcatggaaaaaaaaacaaagaggccaggatgtctggttgttactgccactgtttcacatatccctgaaggacctgcccaatgctaaagaaacacaaggaaggtaagaggtgaaagagaagaaatgaaactatcattgtttgaagatgacaccatcttttacatagaaaacctgttagaatcaaatggcaagctattagaactactaagagaattcagtgaggctgctgtattcatggcaaaattttaacaattgatagcatttctctgcaacattccttaatagttataaaatacagcacaaagtagtaccaaaaatattaactatctaggaaataacctcttacagagaaaatttagtctgttaaaggataaacagtggcaatgtacgtcatgtccacagagattatattttagcttagcaaagataccaattctcccaaatttatttataaattaaatgcaatgtgaatcaaaatttcccactggaatttttatcaggaaggcaacaaattctttctttctttctttctttctttctttatttatttatttatttatttatttatttatttccttccttccttccttccttccttccttcctttctttctttctttctttctttctttctttctttctctctctctttctctctccccccctctctctctctctgtctctctctctctctctttctttctttctttctttctttctttttaagacaaagtctggctctgtcacccaggctgcagtgcagtgatacaatctcagctcactgaaacctcaacctctccggcatcaggtgaacctcccacctcagccccccgagtagctgggactacaggtgcacaccactgggcctagataactttttgtatttattgtaaataaacacaaaaaataaatattttgctcaggttggtctggaactcctgggctcaagcaatccgcctgccttggcctcccaaagtgctagaattacagttgtgagccaccacacccagccaataaattaattctttatgatgaataagttatctatgaaaattaagtcagctgggtgcggtggctcacgcctgtaatcccagcactttgccgggctgaagcaggtggatcacctgaggttgggagttcaagaccagccggaccaacatagagaaaacccgtctctactaaaaatgcaaaattagctgggtgtggtggcatatgcctgtaatcccagatacttaggaggctgaggcaggagaattgcttgaacccgggcggtggaggttgcggtgagccaagattgcaccattgcactccagcctgggccacaagagcgaaactccatctcaaaaaaaaaaaaagagaagttaagtcaatgaaaagttaagtcaattaaaaaagtaagagctgtagtgtttagatatatacacacacacatatatatatatttatctttatatatgtatatatatcttttcctttttttgagaccgagtctgtttttgttgcccaggctggaatgcagtggcgcgatctctgcttactgcaacctctgcctcccaggttcaagcgattctcgtgcctcagcctcccgagtagctgggattacaggtgcctgcccccatgcccggctaatttttgcatttttagtagagacggggtttcaccatgttggccaggctggtctcaaactcctgacctcaggtgatccaccggcctcagcctcccaaagtgctgggattacaggtgtgagccaccgcgcccagccatatattttgcttttcatctgcagctcctggatcctaactccttgttatattgttgggcactttaggcctcagtaaacagaatctctgtctatgaccttctcctgtccttcttccacctgcccaaagcaggactctaatttgattgtgggtcaaaagactctcattccagaaagggccttgcctcataccctagaggaaggaatgctgcacagaaacgccaagtctgaacagacaagccttgctgggtttataccatatgctttttgtccaatcacatttcttcatggttgccaatcatgcctatgtaatgaagcctccataagaacccagaaggacagggttcagagagtttccacatagctgaacactatctggagagtgaacacttcctagagagtggcacacccagagagatcatgaaagctccacgcccctttccccttacctcgccctccacatctcttcatctgtatctttcataatatcctttataaataaaccagcaaatgtgtttccctgagttatgtgagtcactctagcaaattaatcgaacccaaagagggggtcatgggaaccccaacttgaagccagtcagtcagaagttccagaggcccagacttgcaactggggagaaagagggggaggtcttggggactgagcccccaacctgtgggatctgacactgtctccaggtaggtagtgttggaactgcattggaggacactcctggtgtctgctgcttggtgtgtggggggaaaaacccacacctttggttacggaggtcttctgtgttgacgatcattgctgtttgagggcagagggaatacacggtttgagagagtttttccctgacatgagcgaacaggggacatgtactggtctctgagatgggggatcatgggatctgccacaagtggggagaccactgtgacccctgccacagtctttggggcagagggtgtctcgggggcagaagaagcgagagttgtttgcagtagcagttatgtccaaagtgggcgccaggaaagtagggctgcccagctttgaagagcctccttactcccagcctgaatgaaaccatttcctgtaaagcgctaagcataaagtttgccaatggtgatccacggagaagtgagtgtaccccaccccgccatcccacagggaatgtcggagtgatgttgatctgcacctagggaaggaatggttcatgagatgtggtggagatgctgagggcccgtggacatcagatcctaccctacctgtgccaggacaagccatgcgcatgtgcttcagaccaccaggcaacaggagtgttgcatgaggtgtgaagcaggcacctgggaaagaggagtgtgaacagcagatgggacacactgggggcagtcataggaatgaaatgtcccaggatggatgcaggcaggttatggaggacttagtgaggactgctctcctggtgggaattgtggagtgggagactggatggagactggaggtgttttaagtagggaagccaacttgcaagggtgaccagggaaactatgtcggccaagggtgagacatgcactggcaagactctcagacagcctggcttatctaagcagaatgcttgagccatgccaacggtgcctcgcaagttgtattaatcatgtcctttcattttgtgtttttggtgcttggcatctgggcccttgctgaccctaagggaccatttctctcagagctagtcaagtcctagacacagtaaatgactctcctgggagcatgccttccatgtgcagaccaaccaatcaagagtccacactcccacccacctcctttatcgagctctcacatcctggggcaccatccacctgccctaatcactcaaggaccacgtcccaaacaactagggacagcctccatgcccctgcacccattgaaattattcatgctagccaatcctaaacctgtgtatgctgccacaccattccttcctgcagaaacacagtaaggactcttcctacacctcccctacttcctctgctccctgacttacccacttacttcctggtgcagtcccctgtggcatagttcactctcttcttttgggaactgtgaggctatcttctcaatggcagtcatctcctgagctgttggccttgccatacctaactaataataaaatctatattctaaggtaaaaacaaaacagatagggtctcactctgttgcccaggctggagtacagtggtgtgatcatgactcactgcagcctcaaactcctgggctcaagcagttctctcatctcaacctcccgagtagctgggactacaggcacacaccaccatgcctggctagttttcttattttttttgtagatacagggtcttgttatgttgccaaggctggtcttgaactcctgggctcaagtgatcctcctgccttggcctcccaaactgctgcaattacaggcatgagccaccatgcccagatcagaaatcttactaaaaatatttcaaggagaagagaaagccaaagatgttgaatatatatatatgtgtgtgtgtgtgtgtgtatatatatgtatatatgtgtatatatgtgtgtatatatatatgtatatatgtatatatatatgtatatatgtatatatatatgtatattggggcaggcgtggtggctcatgcctgtggtcctaactacttgagagtctgaggtgggaggattgcttgagcctgggagatcgaggctgctgtgagctgagactacaccactgcactccagcttgggtgacagagtgagaccctgtctccaaaaaaacaaaaagaaaaagaaaaaaagatggaaaaagacatgaaaaaacaacaacagaaatacccacacatcatcaatgggagggaagcatcttgaggcagcaaagcgggagtgctagtagagaggcagatagggcgttggacctgaggcattaaggaaagtcaggatttggagcttacaagtctctcattggagatgggatggggttggaatgaatgtctgagcaaacacaaagcatttccttccctaatgactccccaccagtctaaagaatcccacattaggtcgaacacggtggctcacgcctgtaatcccagcactttgggaggccaaggcgggtggatcacgaggtcaggagatcgagaccatcttggctaacatggtgaaaccccgtctctactaaaaatacaaaaaaattagccgggcgtcatggtgggcgcctgtagtcccagctactcgggaggctgaggcaggagaatggtgtgaacccgggaggcagaacttgcagtgagcctagatcgcgccactgcactccagcctgggggacaaaacgagactctgtctcaaaaaaaaaaaaaaaaattcccacattagagttggggaaatgggcagtcctggtggaagttagggaacagatctgggacacgttatagccagctggactacaggaggccataagctcaattcttccttgactctgaaaccttccactggtcctaatgcctagtaattccaggcctttcccagttgtgccaggcttggaggtgaacacatctatgtgccaagaaggaaaggtatgccaagcaggggcttaagtcatccttatcctcagtctgtctatgagtggtatgtacccctgttccccttgcaagatctgctgggcttaggtctcctggctgtgagttccccatacctgggcataaatgtagtgagcctgagctcccaaataaggttgggggctccagagaggtggagagccctgtgtctgggaagtgtgcccacccagcaggtctgaccaggaagatacactgctagggttatggaaaaagactatgtgtcaaggtctcttgattctccatctaggcagagaatcatctttaattaatgggaaactggaaggcaaattacttggacctgaaattactttttgtttattgaaccactgtgttgtaaatcacatctctctgaaggcaagagaaatcagggagttacaaaatgtttaggagaactaaacaggactccctgttttgctaactaatcagattgagacaggctctctggtaaatctacaaatttgatgttgttcaaccataagcagtaaatttcctatgctggattttcctgacaatgaatgtaaaaggaaaaggagtctttttgacaaaatattttattgttcatctaaactgaaaaacttctctatttttcaaaattgctatacgtgtttaaagatgtagatatttgaatagcctaactggtacagaaggtttaatgatgattcctaagacatacctataaattacttgaaattgaaacgaaatttaagaagaattattggaattttccccttctcaaatgagttcttagtttcataaatactatacaagtccataagagatttggggttttgagatgtctttttttttttttttttttcagacggagtttcactgttgttgcctaggctggagtgcaatggcgtgacctcagctcactacaacctccacctcccaggttcaagcgattttcctgcctcagcctcccaagtagctgggattacagggacctgccacaacgccaagctaatgttttgtatttttagtagagatggggttcaccatgttggccaggcttgtctggaactcctgacctcaggtgatccacccgcctataatttattactcccttttgcaaatgtttgaaaaggaataaagtgcaatatttttaaacagaatgcagagttctgttgtcctttggcaataccagtttcagactctgagagtggctcttgctgttgccgacagtgggctgatgaccaaatcccaacatgcccccgctgcgagtccttcataacctgattcagtcatcacttagaggccagcaggcttcagggaggcgtgagcctcagccaacaacctataggggaagagacgcagaactcaatgcagacaggtttggattctggtgcctagagaatgcaacttggaaactctgagccaggagaaaagggttctctctccatgagagagtgtgggctttgtgagaagcgacacacagcaaacacaattaagagtccacccctcagcggggcgcaggggctcacgcctgtaatcccagcactttgggaggccgaggcgggtggatcacgaggtcaggagatcaagaccatcctggctaacacagtgaaaccctgtctctactaaaaatacaaaaaaattagccgggcgtggtggcgggcgcctgtggtcccagctactcgggaggctgaggcaggagaatggtgtgaacccgggaggtggagcttgcagtgagccgagatcgcgccactgcactccagcctgggcgacagagcgagactccatctcaaaaaaaaaaagaaaaagaaaaagaaaaagagtccgcccctgaattaaatagttggtccttttgtgttcctggtgattcacttgctaagtggaagaaacaggagggaatcttttctcctgccctcctg

SEQ ID NO: 14 - DNA, 1209 bp, homo sapiens
cagggctgggcggcggcggcggcggcggtcatggaacgccaagaggagtctctgtccgcgcggccggccctggagaccgaggggctgcgcttcctgcacaccacggtgggctccctgctggccacctatggctggtacatcgtcttcagctgcatccttctctacgtggtctttcagaagctttccgcccggctaagagccttgaggcagaggcagctggaccgagctgcggctgctgtggaacctgatgttgttgttaaacgacaagaagctttagcagctgctcgactgaaaatgcaagaagaactaaatgcgcaagttgaaaagcataaggaaaaactgaaacaacttgaagaagaaaaaaggagacagaagattgaaatgtgggacagcatgcaagaaggaaaaagttacaaaggaaatgcaaagaagccccaggaggaagacagtcctgggccttccacttcatctgtcctgaaacggaaatcggacagaaagcctttgcggggaggaggttataacccgttgtctggtgaaggaggcggacttgctcctggagacctggacgcagaggcccgtcatctggcggatgaggctaagaatcttgttagtgtcacttttgacattagcaagatgaacccttaaccctcgattcaattgccttacgcacgcttttcacagtgactagccaaggggaggtggggttgatttctgttcctaactacacctgcatatgtcagggctccagtcagcaaaaggtatagatgttgcctctaggcatgaggtcattggtcacattctacttggagacagtgattgcattcattgatttcatggttaattgctagttggtaggtaaaggcctctagatgattagcaatcttgataaaagaggcctagtaatgttcttttgaggttagaaatccttgctgctaggacagtctctgtgacaggttgcgttgaatgatgtcttccttatcaatggtgagcccaccagtgaggattactgatgtggacagttgatggggtttgtttctgtatatttatttttatgtacagaactttgtaaaaacgaaactatttaaaaaacaagaataacatttttagcatctttattcaaggagatttatggacttcaatttgtctatcaaacattaaatagctttttattacaacctccaaaaaaaaaaaaaaaaaaaaaaaaaaaaa

SEQ ID NO: 15 - protein, 204 aa, homo sapiens
MERQEESLSARPALETEGLRFLHTTVGSLLATYGWYIVFSCILLYVVFQKLSARLRALRQRQLDRAAAAVEPDVVVKRQEALAAARLKMQEELNAQVEKHKEKLKQLEEEKRRQKIEMWDSMQEGKSYKGNAKKPQEEDSPGPSTSSVLKRKSDRKPLRGGGYNPLSGEGGGLAPGDLDAEARHLADEAKNLVSVTFDISKMNP

SEQ ID NO: 16 - DNA, 10907 bp, homo sapiens
tgagcagcctgagatgtcagtaattgtagctgctccaagcctgggttctgttttttagtgggatttctgttcagatgaacaatccatcctctgcaattttttaaaagcaaaactgcaaatgtttcaggcacagaaaggaggcaaaggtgaagtccaggggaggtcaggggtgtgaggtagatgggagcggatagacacatcactcatttctgtgtctgtcagaagaaccagtagacacttccagaattgtcctttatttatgtcatctccataaaccatctgcaaatgagggttatttggcatttttgtcattttggagccacagaaataaaggatgacaagcagagagccccgggcaggaggcaaaagtcctgtgttccaactatagtcatttctttgctgcatgatctgagttaggtcaccagacttctctgagccccagtttccccagcagtgtatacgggctatgtggggagtattcaggagacagacaactcactcgtcaaatcctccccttcctggccaacaaagctgctgcaaccacagggatttcttctgttcaggtgagtgtagggtgtagggagattggttcaatgtccaattcttctgtttccctggagatcaggttgcccttttttggtagtctctccaattccctccttcccggaagcatgtgacaatcaacaactttgtatacttaagttcagtggacctcaatttcctcatctgtgaaataaacgggactgaaaaatcattctggcctcaagatgctttgttggggtgtctaggtgctccaggtgcttctgggagaggtgacctagtgagggatcagtgggaatagaggtgatattgtggggcttttctggaaattgcagagaggtgcatcgtttttataatttatgaatttttatgtattaatgtcatcctcctgatcttttcagctgcattgggtaaatccttgcctgccagagtgggtcagcggtgagccagaaagggggctcattctaacagtgctgtgtcctcctggagagtgccaactcattctccaagtaaaaaaagccagatttgtggctcacttcgtggggaaatgtgtccagcgcaccaacgcaggcgagggactgggggaggagggaagtgccctcctgcagcacgcgaggttccgggaccggctggcctgctggaactcggccaggctcagctggctcggcgctgggcagccaggagcctgggccccggggagggcggtcccgggcggcgcggtgggccgagcgcgggtcccgcctccttgaggcgggcccgggcggggcggttgtatatcagggccgcgctgagctgcgccagctgaggtgtgagcagctgccgaagtcagttccttgtggagccggagctgggcgcggattcgccgaggcaccgaggcactcagaggaggtgagagagcggcggcagacaacaggggaccccgggccggcggcccagagccgagccaagcgtgcccgcgtgtgtccctgcgtgtccgcgaggatgcgtgttcgcgggtgtgtgctgcgttcacaggtgtttctgcggcaggtgaatgacgggcgtgggtcggtgcgcgctcggcttgcgcacacggtgtctctataagtgcgcgggtgacgagagtcgggatgtgccggagaccccggggcggagagcgggattacaagtacaggaatccctggtcacgctccccgcccctggaaacccagctggggcgagggagggcgtggacgggaccgttctgggagctcgcctttggctgcggttggctccaggccccaggcgcagtttgctcgcggcgtggggatgaagtccgtgtccctggaggggcccaggaagggcgaggaaagcggagtggagtaagttcgtctaggatcggtcccgggtggctctgggatccaatctgcgccgccctggcccaggtcccaggttcaggtcctttacgccactgtgtccaccacctggctgagcgctgaggtcagcgcgggctgtttcctggcccttgggaatgtgccaggacccgtcccctaaggactagcgaggaggtgactcactgtgacaaggagaccccagggaacggactgtatgaggtcagaaccccgcccgggatggggtacagcgggactccagaagccctctcccctgccccttcgcggtctccgtcctcccatcggcacagtgacctatttggctggaacagtttgttcccaaggaagccgggcactggaggtccgggacaccgcgtcgggtccccgctccgcggcgcgctgtaggggtcggggagtcacggccctgcgctgggcgggctctaaccagcctgtcagtcggggaagggcaagggtctcctctacctctttcccaccgcggccgggagaatcgcggcccagcctgtcctcgggtcggggcgctggactccggggcgggagcggagcccacgcctggatgggaggcggggagggttcatgtctttgaggggtggggggtctggggggcacgacgctgctcagggcctctatcagctgcctcgggggctcagggcttcccgacctagcccagattccctctccgaaagctacagggctgagcggagcaggggggcgagtcgccccctggggcgccgccgcctggcgcggaccacagcgcgtcctctccgtcccaaacccctgggggacacttgcgccctcttcgtgaggaaaagcatcttggagctgggttaggaacttggggcgcccaggcagcttcccctctccttgcctccctccacgtcgcgtttctgggaggacttgcgagcggttttgttttcgttgctcccgtctatttttattttccagggatctgactcatcccgtgctttgggcgtggagataaggtggaggggccggctcccggcgcgcgcgcgcgtgcgtgtctgcgcgggcgtgtgtgtgtgtgtgtgtgtgtgtgtgtgtgtgtgtgtctgtgtcagagacggcacaagagcgcgcggtttcccaacagcggcgggagtttcggaagcctggccggctcagcgtgacgtgttcgcggccccccggtcccctcccattctccccctccccaccccagggtgacgcgcagccggagtggaagcagagttttggcgggcgagcagcgccttgcaggaaactgactcatcactactccctccagcggtccgaggctctgcccacgcacctcccactccgcgcgtgatttcctggaggccggcgccccctcccggccctggcgggaatagcacacaggctttcccgcggagtggggctggccggcgcgaaccgccgcggctactcctgggctcatccgagatcaacccctatgccattaccaccccttcaaaggagcactccttaggttcaacagtattcactgagctcttactggaaattaaaatatggctgaagtctaaggcaggaaggccaataaaggaggctatttttaattgtttctaaaacaagggtttgcgtttctgagttttctttgggctgaaagttattatgagcatgagagcagattttgatgggggaggagaggcctatgagagccataagagaaggaggggtggtagaagaggagagggtgcctgcctagatcctagtcctgtcttgaactcccgagagccagggaatatccagctccttgatgaagccctaggcgggcgcctcctccttgtgcctatgatgtattgagacccagaatgtccatttcaaacataccagtgtgtctccgcttggctggcaccccaagagtgcccatctgaggaattgtgccaaacacttgcttgaatcttcaatttggattaagttggtctcgggaggcagggcctcagcaatctatattttgaaaaaactccctaggtgcttttctttctttctttctttctttctttctttctttctttctttctttctttctttctttctttctttctttttctttctttctttctttttctttcttttctttctttctttctttctttctttctttctttctttctttcttcctttctctttctctctttctttctctttctctctttctttcttttctttcgacagagttgcactctgtcacccaggctggagtgcaatggcaccatcctggactcaagtagtcctcctgtttcagcctcccaagtaaccgggaccacaggcgtgatccccccgcccccatgcccagatttttttttttttttttttttttttgagatgcggtctcgctctgtcacccaggctggagtgcagtggcgtgatctcggctcactgcaagctccgcctcccgggttcacgccattctcctgcctcagcctcccgagtagctgggactacaggtgctgccatcatgcccggctaatttttttttgtatttttagtagagacgggggtttcaccgtgttagccaggatggtctcaatctcctgacctcgtgctccgccctcctcggcctcccaaagtgctgggattacaggtgtgagacactgcacccaaccacccagctaatttttatttatttttatttttagtagagacagggtctcagctagttgcccaggctagtcttggacccttgggctcaaatgattctcccacctctgcctcccagagtattaggattacaggcataagccactgcccctggcctccccaagtgattgtgatgggcctctctggttaagaaacctcaaaattagagagggagtggggttcaatactacagcacaggactcagggcaaacaggcctgggttcagatcctggctgtgccacttatgaactgtgtgatgttaggcaagttacttaacttatctgagccttggttgcctcttctgtaaaaagggagctaatagatatccactttttaggaggattgatatttttaaactgcttagaacagcccccaaacataaaaatatataaataaatcccaactcatgcctagcagagggtggatagaggttatttgagggctctgtccactgtactgggtgacccctttatggggcagtggcctttggcctttttagctgtatgactcaggggcaagtctcatatctcttccatctcctgccctttaaacttggtgtgaagttaccaagagcctcctctcccaaccagctgggacgtgaaactgtgggctccactgatcacaagcagtggggtgaggtggggtggagcagatgtggcatgtgtcccgggcttcctgcctcatgaggactcagcagagctttcacccccagaaactgcaagttgggacttgtccctaggaaaatccagttgctgccaaggtcgtgcagtcactcagccctggagtcaagccagagcaggcaggtaggtgccagggctccctcatgggcaaactcactctccgttttccctctcctgaagggggaggagaggagccaggtagaccagccacctttaattttctttttgcctgcaaaacggtttccttggacacaggcaacacgaggcaggggctgccaggtgtctagacttcagatcacctgatgtgcctggcaggatgtggctcagcctgggagaaatcatcccttgcgctgccccgcccggcccctccttacccctaggccacccgcctgacgacatccttgggaaaggccctcagcctacagcacctgtcagctgctgtctgaaggaggtagttggcagggggaagtgataggggggaggctcagtaaaactgaaggcagagaggaataatcatacttctgttttcaatgcacttctctatacgaagtgctgctggcacgttacctacattaactcagttaattctcatgtctatcctctgagacagtcactattactatccccattttatagatgaggaaactagagctcagacaagttaagttgcttgcccagggtcacctagtaaaacctggactccagcccaggtgatctggctccagagccctcctgcttaaccaccaggatacagcctttcattcagctctgttctgtctgccttgctgcatggactctgtgatcaatttcttgagtatgtgtctgtagccatgctctttaaacttgtacatggccccatttatggatgaggaaactgagacctagagacattaagtggctttttaaagcttacgtagtaactggcagagctaggaccacaacccgggtgctttttgccccaaagtcccgggtacttttacttggcagagcagggttaccctacttggggatctgggtcgggggacttaggaggctggaggaactgtcagactgtttcttcttttgggaattgaccttctggccagggctgcgattaggaaactgctggactctggcaattcacacatatttggggggcattcacacccatgagggacacctctggggggaaaacaaattgattttagctgataatacctggtggcaaacaggaccctggtccttgctcttgcaatagacttgcctttgttgacattagcttgcccttcagttgcctgctctcccagtgaccttggtgtgccaggctggctgagctctgctggtgggggtcaggcctcctgtgggaaggaagcaggaagaccagctggaaggagtgagagagaccctctggtaggaagacgtcacctgaggtgacacagcaaagcccggccaggtaacatagtgtctaatctccgccgtgaccagggccttccttgtatctctgctgcaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacaggtgcggacatgtgcacggaaggactttgtaagggaccaggattctcagaatccatggtccaagggctgacctgtctggtcctggtccagcatgctccaggtagaaggaaacaggcccagagaggggaagcaacctccctgaggtcacacagcaagtaggcagcaaagaccaactagctaacatttattgggaatgttcattatgccaggccctttgccaagcttctaaggtagatttatttagtccttatagcaatgttataacataagacattcttgtcaccctgcccgcctttctttttgagacaggtgtcttaactctgttggccagactggagtgcagtgatacgatcatggctcactgcagcttcaaactcctgggctcaagcgatcttcctacctcagcctcctgggtagctgggaagctgggactatagttgtacaccactacgcccggttaattttttgagtttttgtagagacaaggtctcaccatgttgcccgggctggtcttgaactcctgagctcaagcagtcctcctgcctcagcctcccaaagtgttgtgattacaggcgtgagccaccatgcccagccccttgccatccttttagggcaaggaaaccaggctcagagaggtagagtgatttatctaaggtctcaaagtgaatttgccgttgggtcaagactaattataataacaacaactactgacgtttatatgggcccggcattgtgctgaacactttcatggattttgtaacagaatccctagatcagcactgtccagtaactctgcagggatgggagtgtccggtacaggggccacgagccacatacggctgttgtgcatttgacacacagctcatgtgactgaggaactgaattgttcattttatttgattgtagtctgtttaaacaagcacacagagctagtagtggttcctctgctgggcagcttgacttagagcagacccatgggtgcgggtgcggtgatggataaaatcacatctgtgaagcatggtgggacactccataatacccctcaagagacagagtggacgttccccgagttcttcctgttctcagcagtcggccccattggccccagggaagggtgtcctggccccccactgtcttcctcagttgggcagctccgccgcgtcctcttcttcttggcctggctgacttctgctgtctctcctcagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaatattactattgtggttattataccttataagaacaaataaatgggcttttgggaaggatttcataattaaataattttaaaaattaagcatttaaatttagagaatgcagaaaacttagcaaacagaaagactgctgcaaaaaacaacagcaaaacaaaaactactgtcacacctctgcaaagatcaccaatgtcaatattttggtttgttgtgtaatctttttgtaaagaatatattatagcttaacatcattattcatcagataaatgcaaattaagataccacaataagataccaccatacacttaccagaatgattaaaaaagactgacagtgccaagcattggcaaggttatggagcaactggatctcttatttaaaaaaactgtttgggccgggcgcagtggctcacacctagaatcccagtgcttcgggaggctgaggcaggagatcacttgaggccaagggttcaagaccagcctggccaacatggtgaaatctctactaaaaatacaaaaattagctgggcatggtggtgcacgcttgtaatcccagctacttggaaggctgaggtgggaggatcacttgaacccaggaggcagaggttgcagtcagctgagatcataccactgtactccagcctcttccagggtgacagtgagattcatctcaaataaatacataaataaaaaactgtttggtaatatcttctaaagatgcctaccttcatggctacctcatgacccagtaattctattcctggacatgttctcgagagaaatgagttcatatttccactgaaaaaggcataagaatgttctacacagtggctcacacctataatcccagcactttgggaggctaaggcaggaggacggcttgagcccaagagtgtgagaccagtttgggcaacatagcgagactcttatctc

SEQ ID NO: 17 - protein, 164 aa, homo sapiens
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP